(19) United States
(12) Patent Application Publication
(10) Pub. No.: US 2006/0009423 A1
(43) Pub. Date: Jan. 12, 2006

(54) Phosphonate-phosphate and diphosphonate apolipoprotein e modulators

(76) Inventors: Eric Joseph Niesor, Nyon (CH); Craig Leigh Bentzen, Bogis-Bossey (CH); Lan Mong Nguyen, Nyon (CH)

Correspondence Address: MCDONNELL BOEHNEN HULBERT & BERGHOFF LLP, 300 S. WACKER DRIVE, 32ND FLOOR, CHICAGO, IL, 60606, US

(21) Appl. No.: 10/505,045

(22) PCT Filed: Feb. 14, 2003
(86) PCT No.: PCT/US03/04706

§ 371 Date: Aug. 19, 2005

Related U.S. Application Data
(60) Provisional application No. 60/358,130, filed on Feb. 19, 2002.

(51) Int. Cl. A61K 31/66

(52) U.S. Cl. 514/130

(57) ABSTRACT

The present invention relates to methods of use of phosphonate-phosphates and diphosphonates to modulate apolipoprotein E levels and the use of such compounds in therapy, including cardiovascular and neurological disease states.

### FIELD OF INVENTION

The present invention relates to phosphonate-phosphates and diphosphonate compounds, the processes for their preparation, pharmaceutical compositions containing them and their use in therapy, in particular for modulating (increasing and decreasing) apolipoprotein E in plasma and in tissues.

### BACKGROUND OF THE INVENTION

Apolipoprotein E (apoE) is a polymorphic, multifunctional protein synthesized by several cell types and tissues, including liver, kidney, skin, adipose tissue, macrophages and brain. The wide distribution of apoE is associated with the maintenance of key cellular functions such as intracellular cholesterol trafficking, cholesterol distribution between cells, and tissue reparation.

The amino acid sequence of the apoE protein is well conserved throughout species. ApoE can be viewed as a regulator of cholesterol homeostasis in tissues such as the central nervous system (CNS) and peripheral nervous system (PNS) and the arterial wall (cell-cell) or between tissues via the circulating plasma lipoproteins (tissue-tissue).

The major role of plasma apoE containing lipoproteins is to transfer lipids (cholesterol) from peripheral tissues to the liver and to remove excess cholesterol from peripheral tissues via the reverse cholesterol transport system. Dysregulation of this mechanism leads to excess cholesterol deposition in peripheral tissues such as arteries (arterosclerosis) and skin (xanthomas and xanthelasmas). ApoE has also been shown to have a direct effect on lymphocyte proliferation and thus has an immunomodulatory role.

ApoE is the only lipoprotein synthesized in the brain and has a key role in cholesterol transport between cells of the CNS. Local secretion of apoE by cells such as macrophages or macrophage-derived cells is essential for the uptake of excess tissue cholesterol and the provision of cholesterol for specific needs such as nerve repair and remyelinisation.

Up to the present time, compounds affecting apoE production in vitro and in vivo have not been extensively investigated. Only hormone-like estrogens and corticoids have been shown to change apoE levels under various experimental conditions (Srivastava et al., 1997; Stone et al., 1997).

There is currently a need for compounds that modulate apoE synthesis and secretion, such compounds having application in the treatment of diseases such as atherosclerosis, excess lipid deposition in peripheral tissues such as skin (xanthomas), stroke, memory loss, optic nerve and retinal pathologies (i.e., macular degeneration, retinitis pigmentosa), repair of traumatic damage of the central nervous system (brain tissue), repair of traumatic damage of the peripheral nervous system (i.e., nerve section compression or crush), prevention of the degenerative process due to aging (i.e., Alzheimer's disease), prevention of degenerative neuropathies occurring in diseases such as diabetic neuropathies and multiple sclerosis, autoimmune diseases and activation of the innate immune system.

### SUMMARY OF THE INVENTION

As shown herein, certain phosphonate-phosphates and diphosphonates modulate (increase or decrease) the production of apoE in vitro and in vivo. Thus, one aspect of the present invention is a method of modulating the production of apoE by an apoE producing cell comprising contacting said apoE producing cell with an effective amount of a compound of formula (I): 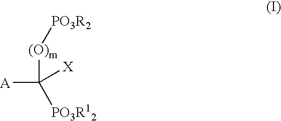
wherein X is H, OH, OCOCH₃ or NH₂; R and R¹ are the same or different and are selected from H, CH₃ or C₂H₅, n- or i-C₃H₇, n-, i- or s-C₄H₉; m is zero or 1; and A is selected from: 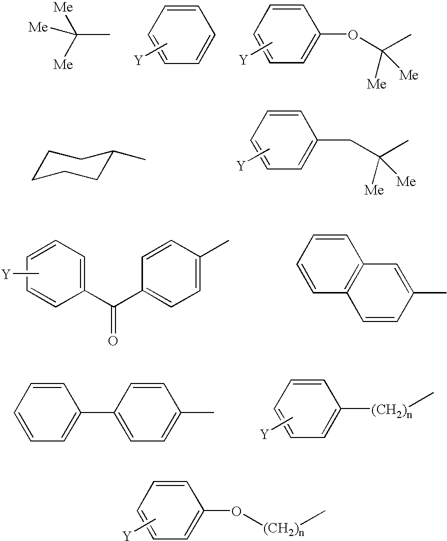
where n is an integer from 1 to 6 and Y is H, CH₃, OCH₃, CF₃, Br or Cl; or a pharmaceutically acceptable salt thereof.

In various embodiments of the present inventions, the compound of formula (I) is a hydroxydiphosphonate wherein X is OH and m is zero; a phosphonophosphate wherein X is H and m is 1; or a diphosphonate wherein X is H and m is zero. In one embodiment, X is OH, m is zero, A is 4-chlorophenyl and R and R¹ are each methyl.

In other embodiments, the compound of formula (I) is selected from the group consisting of:

dimethyl α-(dimethoxyphosphinyl)-p-chlorobenzyl phosphate;

dimethyl α-(dimethoxyphosphinyl)-o-chlorobenzyl phosphate;

dimethyl α-(dimethoxyphosphinyl)-m-chlorobenzyl phosphate;

dimethyl α-(dimethoxyphosphinyl)-2,4-dichlorobenzyl phosphate;

diethyl α-(diethoxyphosphinyl)-p-chlorobenzyl phosphate;

diisopropyl α-(diisopropoxyphosphinyl)-p-chlorobenzyl phosphate;

dipropyl α-(dipropyloxyphosphinyl)-p-chlorobenzyl phosphate;

dibutyl α-(dibutyloxyphosphinyl)-p-chlorobenzyl phosphate;

dimethyl α-(dimethoxyphosphinyl)-benzyl phosphate;

dimethyl α-(dimethoxyphosphinyl)-p-methylbenzyl phosphate;

dimethyl α-(dimethoxyphosphinyl)-p-methoxybenzyl phosphate;

dimethyl α-(dimethoxyphosphinyl)-p-fluorobenzyl phosphate;

dimethyl α-(dimethoxyphosphinyl)-m-bromobenzyl phosphate;

dimethyl α-(dimethoxyphosphinyl)-p-(trifluoromethyl)benzyl phosphate,

dimethyl α-(dimethoxyphosphinyl)-4-(4-chlorobenzoyl)benzyl phosphate;

dimethyl [1-(dimethoxyphosphinyl)-2,2-dimethyl-2-phenyl]ethyl phosphate;

dimethyl [1-(dimethoxyphosphinyl)-2,2-dimethyl-2-(p-chlorophenyl)]ethyl phosphate;

dimethyl [1-(dimethoxyphosphinyl)-2,2-dimethyl-2-benzyl]ethyl phosphate;

dimethyl α-(dimethoxyphosphinyl)cyclohexylmethyl phosphate;

dimethyl α-(dimethoxyphosphinyl)-2-naphthylmethyl phosphate;

dimethyl α-(dimethoxyphosphinyl)-4-biphenylmethyl phosphate;

tetraethyl 2-(p-chlorophenylethylidene)1,1-diphosphonate;

tetrabutyl 2-(p-chlorophenylethylidene)1,1-diphosphonate;

tetramethyl 2-(p-chlorophenylethylidene)1,1-diphosphonate;

tetramethyl 1-chloro-2-(p-chlorophenylethylidene)1, 1-diphosphonate;

tetraethyl 2-(phenylethylidene)1,1-diphosphonate;

tetraethyl 3-(phenylpropylidene)1,1-diphosphonate;

tetraethyl 4-(phenylbutylidene)1,1-diphosphonate;

tetraethyl 3-(phenoxypropylidene)1,1-diphosphonate;

tetraethyl 4-(phenoxybutylidene)1,1-diphosphonate;

tetramethyl 2-(phenylethylidene) 1,1-diphosphonate;

tetramethyl 3-(phenylpropylidene)1,1-diphosphonate;

tetramethyl 4-(phenylbutylidene)1,1-diphosphonate;

tetramethyl 3-(phenoxypropylidene)1,1-diphosphonate; and

tetramethyl 4-(phenoxybutylidene)1,1-diphosphonate.

The method provides that the modulation of cellular apoE production comprises increasing or decreasing the production of apoE.

Another aspect of the present invention provides for a method of modulating apoE levels in a patient in need of such treatment, comprising administration of an effective amount of a compound of formula (1) as described above. Modulation of apoE levels includes modulation of plasma and/or tissue apoE levels. The method provides that the modulation of the apoE levels comprises increasing or decreasing such levels.

Wherein the levels of apoE are increased, the method provides that the patient may be suffering from atherosclerosis, Alzheimer's disease, macular degeneration, retinitis pigmentosa, stroke, xanthoma, xanthelasma or degenerative neuropathy, wherein the latter may be associated with diabetic neuropathy or multiple sclerosis.

Other aspects of the present invention provide for methods for elevating high density cholesterol, preventing and/or treating atherosclerosis, preventing and/or treating macular degeneration and retinitis pigmentosa, preventing and/or treating stroke, prevention of degenerative neuropathy, comprising administration of an effective amount of a compound of formula (I) as described above.

Embodiments wherein the levels of apoE are decreased include wherein the patient expresses apoE4, apoE Leiden or a non-functional mutant form of apoE and when the patient is suffering from atherosclerosis or Alzheimer's disease.

A further aspect of the invention provides for a method for the prevention and/or treatment of Alzheimer's disease or dementia comprising administration of an effective amount of a compound of formula (I) as described above. In embodiments wherein the patient is heterozygous or homozygous for apoE2 and/or apoE3, the method provides for the administration of an effective amount of a compound of formula (I) wherein the apoE levels are increased. In embodiments wherein the patient is heterozygous or homozygous for apoE4, the method provides for the administration of an effective amount of a compound of formula (I) wherein the apoE levels are decreased.

### DETAILED DESCRIPTION OF THE INVENTION

1. Phosphonate-Phosphate and Diphosphonate Compounds

The present invention relates to phosphonate-phosphate and diphosphonate compounds of formula (I) that modulate apoE levels and are useful as agents for the treatment of a number of disorders including cardiovascular and neurological disease states.

Pharmaceutically acceptable salts for use in the present invention include those described by Berge et al. (1997), herein incorporated by reference. Such salts may be formed from inorganic and organic acids. Representative examples thereof include maleic, fumaric, benzoic, ascorbic, pamoic, succinic, bismethylenesalicylic, methanesulfonic, ethanedisulfonic, acetic, propionic, tartaric, salicylic, citric, gluconic, aspartic, stearic, palmitic, itaconic, glycolic, p-aminobenzoic, glutamic, benzenesulfonic, hydrochloric, hydrobromic, sulfuric, cyclohexylsulfamic, phosphoric and nitric acids.

Since the compounds of the present invention, in particular compounds of formula (I), are intended for use in pharmaceutical compositions, it will be understood that they are each provided in substantially pure form, for example at least 50% pure, more suitably at least 75% pure, preferably at least 95% pure, and more preferably at least 99% pure (% are on a wt/wt basis). Impure preparations of the compounds of formula (I) may be used for preparing the more pure forms used in the pharmaceutical compositions. Although the purity of intermediate compounds of the present invention is less critical, it will be readily understood that the substantially pure form is preferred as for the compounds of formula (I). Preferably, whenever possible, the compounds of the present invention are obtained in crystalline form.

When some of the compounds of this invention are allowed to crystallise or are recrystallised from organic solvents, solvent of crystallisation may be present in the crystalline product. This invention includes within its scope such solvates. Similarly, some of the compounds of this invention may be crystallised or recrystallised from solvents containing water. In such cases, water of hydration may be formed. This invention includes within its scope stoichiometric hydrates as well as compounds containing variable amounts of water that may be produced by processes such as lyophilisation. In addition, different crystallisation conditions may lead to the formation of different polymorphic forms of crystalline products. This invention includes within its scope all polymorphic forms of the compounds of formula (I).

II. Applications of ApoE Modulators

A. ApoE in atherosclerosis

As a component of all lipoprotein fractions, apoE plays an important role in cholesterol homeostasis, by mediating their interaction with receptors such as the apoB, low-density lipoprotein (LDL) and other specific receptors. The important role of apoE in cardiovascular diseases is demonstrated by the apoE knock-out mouse model, where the animals rapidly develop hypercholesterolemia and atherosclerosis with pathological features similar to human atherosclerosis (Plump, 1997). In addition, the absence of a functional apoE in humans is associated with abnormally high plasma levels of cholesterol and triglycerides and the rapid development of atherosclerosis, notwithstanding a low fat diet (Richard et al., 1995). In the knock-out mouse model, these changes are prevented by infusion of apoE, transplantation of macrophage producing apoE, or gene therapy by introducing the human apoE gene into apoE knock-out mice (Linton et al., 1995). These results indicate a direct beneficial role for apoE and, consequently, a utility for compounds that increase the apoE levels. The compounds of formula (I) that increase apoE plasma levels will decrease plasma atherogenic lipoproteins (VLDL, IDL and LDL) by increasing their uptake by the liver. Increasing apoE in HDL will increase the removal of cholesterol from loaded tissues (atherosclerotic arteries) by the reverse cholesterol transport mechanism.

In contrast, hyperlipidemic patients susceptible of developing atherosclerosis due to the expression of a mutated form of apoE, such as apoE Leiden or other variants, should benefit from the treatment with the compounds that decrease apoE production (van Vlijmen et al., 1998; Richard, 1995). Thus, compounds of formula (I) that decrease the production of apoE are useful in the prevention and/or treatment of pathological cardiovascular conditions secondary to the presence of non-functional, variants or mutant forms of the apoE molecule.

B. ApoE in the Central Nervous System (CNS)

ApoE also plays a critical role in the CNS. In the brain, apoE is synthesized and secreted by astrocytes, its principal role being cholesterol transport between cells. ApoE is considered to redistribute lipids and to participate in the cholesterol homeostasis of the brain.

ApoE is linked to the neuropathological lesions characteristic of Alzheimer's disease. One isoform, apoE4, is strongly associated with the age of onset of the disease (Poirier, 1994; Rubinsztein, 1995), while another isoform, apoE3, is believed to help maintain healthy microtubules. The increase in both apoE mRNA and the number of astrocytes in the brains of Alzheimer's patients indicates that increased apoE represents an astrocyte repair-mechanism to ameliorate the damage within the nervous cells. Memory deficit, defective repair of brain injury and deposition of the Alzheimer's associated β-amyloid variant APP^{V717F} have been demonstrated in the absence of the apoE gene, i.e., apoE knock out mice (Oitzl et al., 1997; Laskowitz et al., 1997; Walker et al., 1997).

Thus, there is a benefit to increasing apoe production in patients bearing the E2 and E3 isoforms of apoE in regard to the occurrence of Alzheimer's or other spontaneous or traumatic neurological diseases. The compounds of formula (I) that increase apoE in the brain will prevent the deposition of plaques associated with Alzheimer's disease and increase the repair mechanism of brain injuries due to mechanical traumas or strokes. Through the increase of neurite extension synaptic sprouting the overall brain activity (i.e., memory) should improve.

Conversely, patients at risk of or suffering from Alzheimer's or spontaneous or traumatic neurological diseases who overexpress the pathological isoforms of apoE, such as apoE4, should benefit from the treatment with a compound that decreases apoE. Thus, compounds of formula (I) that decrease the production of apoE are useful in the prevention and/or treatment of the symptomatic and neuropathological cardiovascular conditions characteristic of Alzheimer's or other spontaneous or traumatic neurological diseases that are caused or exacerbated by non-functional, variants or mutant forms of the apoE.

C. ApoE in the Peripheral Nervous System (PNS)

The important role of apoE in nerve regeneration in the PNS is demonstrated by the observation that apoE synthesis is dramatically induced when nerves are injured (Poirier, 1994). The maintenance and/or repair of the myelin sheets involves the participation of apoE secreted by support cells such as glial and Schwann cells. Both apoE synthesis and concentration were found to be abnormally low in degenerative diseases of nervous tissues such as in multiple sclerosis (Gaillard, 1996). ApoE is also considered to stabilize the cytoskeleton apparatus and support neurite elongation, thus having a major effect on the development and remodeling following injury of the nervous system occurring late in life. Thus, the compounds of the present invention that increase apoE will support and increase the speed of the healing process of traumatized nerves (nerve section, crush, etc.) and the prevention and/or healing of degenerative nerves (e.g., multiple sclerosis).

D. ApoE as Modulators of the Immune System

ApoE affects the immune system by acting on lymphocyte proliferation. Furthermore apoE knock out mice are highly sensitive to bacterial infection due to a defect in the innate immune system, suggesting that increasing apoE production should augment the immune response (Roselaar & Daugherty, 1998). Increasing apoE production by utilization of compounds of the present invention should augment ameliorate the immune response in patients in need thereof.

E. Skin Lipid Metabolism Disorders

Lipid homeostasis is well controlled in epithelial cells such as keratinocytes, wherein exported lipids are important for comeocyte adhesion and for forming the cutaneous barrier to the external environment. Excess cholesterol deposition in skin (xanthomas and xanthelasmas) will be prevented by utilization of compounds of formula (I) that increase the level of cutaneous apoE.

III. Formulations and Administration

The compounds of formula (I) can be administered by any of a variety of routes. Thus, for example, they can be administered orally, or by delivery across another mucosal surface (for example across the nasal, buccal, bronchial or rectal mucosa), transdermally, or by injection (for example intradermal, intraperitoneal, intravenous or intramuscular injection).

When the compounds are intended for oral administration, they can be formulated, for example, as tablets, capsules; granules, pills, lozenges, powders, solutions, emulsions, syrups, suspensions, or any other pharmaceutical form suitable for oral administration. Oral dosage forms can, if desired, be coated with one or more release delaying coatings to allow the release of the active compound to be controlled or targeted at a particular part of the enteric tract.

Tablets and other solid or liquid oral dosage forms can be prepared (e.g. in standard fashion) from the compounds of formula (I) and a pharmaceutically acceptable solubilizer, diluent or carrier. Examples of solubilizers, diluents or carriers include sugars such as lactose, starches, cellulose and its derivatives, powdered tracaganth, malt, gelatin, talc, stearic acid, magnesium stearate, calcium sulfate, vegetable oils, polyols such as glycerol, propyleneglycol and polyethyleneglycols, alginic acids and alginates, agar, pyrogen free water, isotonic saline, phosphate buffered solutions, and optionally other pharmaceutical excipients such as disintegrants, lubricants, wetting agents such as sodium lauryl sulfate, coloring agents, flavoring agents and preservatives, etc.

Capsules can be of the hard or soft variety and can contain the active compound in solid, liquid or semisolid form. Typically such capsules are formed from gelatin or an equivalent substance and can be coated or uncoated. If it is desired to delay the release of the active compound until the capsule has passed through the stomach and into the intestine, the capsule can be provided with a pH sensitive coating adapted to dissolve at the pH found in the duodenum or ileum. Examples of such coatings include the Eudragits, the uses of which are well known.

Formulations for injection will usually be made up of the appropriate solubilizers such as detergents which may also include compounds and excipients such as buffering agents to provide an isotonic solution having the correct physiological pH. The injectable solutions are typically pyrogen-free and can be provided in sealed vials or ampoules containing a unit dose of compound.

A unit dosage form of the compounds of the invention typically will contain from 0.1% to 99% by weight of the active substance, more usually from 5% to 75% of the active substance. By way of example, a unit dosage form can contain from 1 mg to 1 g of the compound, more usually from 10 mg to 500 mg, for example between 50 mg and 400 mg, and typically in doses of 100 mg to 200 mg.

The compounds of the invention will be administered in amounts which are effective to provide the desired therapeutic effect. The concentrations necessary to provide the desired therapeutic effect will vary according to among other things the precise nature of the disease, the size, weight and age of the patient and the severity of the disease. The doses administered will preferably be non-toxic to the patient, although in certain circumstances the severity of the disease under treatment may necessitate administering an amount of compound that causes some signs of toxicity.

Typically, the compounds of the invention will be administered in amounts in the range 0.01 mg/kg to 100 mg/kg body weight, more preferably 0.1 mg/kg to 10 mg/kg body weight and particularly 1 mg/kg to 5 mg/kg body weight. For an average human of 70 kg weight, a typical daily dosage of the compounds of the invention would be in the range of 70 mg to 700 mg. Such a dosage can be administered, for example from two to four times daily. Ultimately however, the size of the doses administered and the frequency of administration will be at the discretion and judgment of the physician treating the patient.

For therapeutic use the compounds of the present invention will generally be administered in a standard pharmaceutical composition obtained by admixture with a pharmaceutical carrier selected with regard to the intended route of administration and standard pharmaceutical practice. For example, they may be administered orally in the form of tablets containing such excipients as starch or lactose, or in capsule, ovules or lozenges either alone or in admixture with excipients, or in the form of elixirs or suspensions containing flavoring or coloring agents. They may be injected parenterally, for example, intravenously, intramuscularly or subcutaneously. For parenteral administration, they are best used in the form of a sterile aqueous solution that may contain other substances, for example, enough salts or glucose to make the solution isotonic with blood. The choice of form for administration as well as effective dosages will vary depending, inter alia, on the condition being treated. The choice of mode of administration and dosage is within the skill of the art.

The compounds of formula (I) and their pharmaceutically acceptable salts which are active when given orally can be formulated as liquids, for example syrups, suspensions or emulsions or as solids for example, tablets, capsules and lozenges. A liquid formulation will generally consist of a suspension or solution of the compound or pharmaceutically acceptable salt in suitable liquid carrier(s) for example, ethanol, glycerine, non-aqueous solvent, for example polyethylene glycol, oils, or water with a suspending agent, preservative, flavoring or coloring agents. A composition in the form of a tablet can be prepared using any suitable pharmaceutical carrier(s) routinely used for preparing solid formulations. Examples of such carriers include magnesium stearate, starch, lactose, sucrose and cellulose. A composition in the form of a capsule can be prepared using routine encapsulation procedures. For example, pellets containing the active ingredient can be prepared using standard carriers and then filled into a hard gelatin capsule; alternatively, a dispersion or suspension can be prepared using any suitable pharmaceutical carrier(s), for example aqueous gums, celluloses, silicates or oils and the dispersion or suspension then filled into a soft gelatin capsule.

Typical parenteral compositions consist of a solution or suspension of the compound or pharmaceutically acceptable salt in a sterile aqueous carrier or parenterally acceptable oil, for example polyethylene glycol, polyvinyl pyrrolidone, lecithin, arachis oil or sesame oil. Alternatively, the solution can be lyophilised and then reconstituted with a suitable solvent just prior to administration.

A typical suppository formulation comprises a compound of formula (I) or a pharmaceutically acceptable salt thereof which is active when administered in this way, with a binding and/or lubricating agent such as polymeric glycols, gelatins or cocoa butter or other low melting vegetable or synthetic waxes or fats.

Preferably the composition is in unit dose form such as a tablet or capsule. Each dosage unit for oral administration contains preferably from 1 to 250 mg (and for parenteral administration contains preferably from 0.1 to 25 mg) of a compound of the structure (I) or a pharmaceutically acceptable salt thereof calculated as the free base.

The pharmaceutically acceptable compounds of the invention will normally be administered to a subject in a daily dosage regimen. For an adult patient this may be, for example, an oral dose of between 1 mg and 500 mg, preferably between 1 mg and 250 mg, or an intravenous, subcutaneous, or intramuscular dose of between 0.1 mg and 100 mg, preferably between 0.1 mg and 25 mg, of the compound of the structure (I) or a pharmaceutically acceptable salt thereof calculated as the free base, the compound being administered 1 to 4 times per day.

Disease states which could benefit from increasing plasma and tissue apoE levels include, but are not limited to: atherosclerosis, neurodegenerative disorders such as Alzheimer's disease or dementia. The compounds of this invention modulate apoE and are therefore of value in the treatment of any of these conditions.

Compounds ofthe present invention may also be of use in preventing and/or treating the above-mentioned disease states in combination with anti-hyperlipidaemic, anti-atherosclerotic, ariti-diabetic, anti-anginal, anti-inflammatory or anti-hypertension agents. Examples of the above include cholesterol synthesis inhibitors such as statins, for instance atorvastatin, simvastatin, pravastatin, cerivastatin, fluvastatin, lovastatin and ZD 4522 (also referred to as S-4522, Astra Zeneca), anti-oxidants such as probucol, insulin sensitisers such as a PPAR gamma activator, for instance G1262570 (Glaxo Wellcome) and the glitazone class of compounds such as rosiglitazone (Avandia, SmithKline Beecham), troglitazone and pioglitazone, calcium channel antagonists, and anti-inflamrnmatory drugs such as NSAIDs.

IV. Synthesis of Phosphonate-Phosphates and Diphosphonate Compounds

The compounds of formula (I) may be prepared in accordance with the synthetic processes described in U.S. Pat. Nos. 4,309,364, 4,371,527 and 4,416,877 and European Patent No. 015,370, the disclosures of which are incorporated herein by reference. It will be appreciated that within the compounds of formula (I) there are subsets of compounds, namely hydroxydiphosphonates wherein X is OH and m is zero, phosphonophosphates wherein X is H and m is 1, and diphosphonates wherein X is H and m is zero.

### EXAMPLES OF THE INVENTION

Without further elaboration, it is believed that one skilled in the art can, using the preceding description, utilize the present invention to its fullest extent. The following specific examples are intended merely to illustrate the invention and not to limit the scope of the disclosure or the scope of the claims in any way whatsoever.

### Example 1

### Biological Activity—Plasma Levels In Vivo

A convenient model for the screening of apoE modulating compounds is the model described in Leininger-Muller & Siest, 1996. This model involves the use of rats fed a normal diet. Selected compounds of formula (I) ae submitted to the in vivo rat test. Following are the results for compound 1, diethyl α-(dimethoxyphosphinyl)-p-chlorobenzyl phosphate:

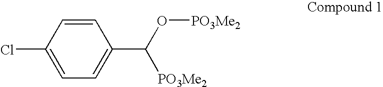

Two groups of 4 or 5 OFA rats (Iffa-Credo, France) weighing between 140 g and 160 g were acclimatized for at least one week, allowed UAR food and tap water ad libitum, under a 12/12 hours light/obscurity cycle (7.00 am-7.00 pm) then treated daily between 3.00 pm and 4.00 pm with test compounds (200 mg/kg/day) p.o. in a vehicle volume of 1 ml for 5 days. Compound 1 was given as a suspension in 20% Tween-80 supplemented with 0.5% carboxymethylcellulose. Control group received 1 ml vehicle alone. After a treatment period of 5 days, rats were weighed, sacrificed by decapitation under pentobarbital anesthesia after an overnight fast. Blood was collected on EDTA and plasma was used for analysis. When given orally to rats at the dose of 400 mg/kg per day for 5 days, compound 1 increased plasma apoE by 61±8%.

### Example 2

### Biological Activity—Sciatic Nerve Tissue Levels In Vivo

A rat model of sciatic injury was established according to Goodrum & Boudin, 1996. Rats with or without nerve injury were treated orally with compound 1 (400 mg/kg for 5 weeks). The amount of apoE was determined in homogenates of the sciatic nerves by ELISA using a goat anti-apolipoprotein E antibody. A dilution to 1/200 of rat plasma was coated on microtiter plates; apoE was recognized by the polyclonal antibody; the conjugate (anti-goat IgG peroxidase) antibody was then added for substrate biochemical recognition. Apolipoprotein E was expressed as % change from mean control value. Cholesterol was measured with a commercially available enzymatic kit (Bioreac, Switzerland). Results were expressed as % change from control group.

Compound 1 administered orally at 400 mg/kg/day for 37 days increased apoE by 154% in the crushed sciatic nerve and by 58% in the contralateral nerve.

### Example 3

### In Vitro Biological Activity

The in vitro assay comprises determining the effect of a compound of formula (I) in modulating the secretion of apoE by an apoE secreting cell line (e.g., a monocyte-macrophage cell line such as the THP-1 cell line, a liver derived cell line such as the HepG2 cell line, an intestinal derived cell line such as the CaCo2 cell line, or a brain derived cell line such as the astrocytoma CCF-STTG1 cell line). The experimental details for use of the THP-1 cell line in the in vitro assay are provided below.

(a) Cel Culture

The THP-1 cell line was derived from the peripheral blood of a 1-year-old boy with acute monocytic leukaemia and were obtained from the European Collection of Animal Cell Cultures (ECACC, #88081201). These cells do not express surface and cytoplasmic immunoglobulins; are phagocytic and differentiate into macrophage-like cells. The cells are grown as non-adherent cells in RPMI 1640 culture medium, 2 mM glutamine, 20 μM 2-mercaptoethanol. Fresh medium is added to maintain cell density between 2 and 9×10⁵ cells/ml. Once a week, new cultures are initiated by inoculating 10 ml of medium with 2×10⁶ cells in a 75 cm² plate. The plates are kept at 37° C. in a 5% CO₂ atmosphere. For screening, cells are seeded in 24-well plates at the density of 2×10⁵ cells per well. Phorbol-12-myristat-13-acetate (PMA) is added at 0 and 5 nM to initiate THP-1 differentiation into adherent macrophage-like cells. Vehicles, reference compounds and test compounds are added simultaneously at concentrations varying from 50 μM and incubated for 72 hours. The culture medium is then recovered, centrifuged at 300 g for 5 min to remove any unattached cell and stored at −20° C. before analysis.

(b) ApoE Determination by ELISA

Ninety-six well-microtiter plates are coated by incubating with a 5% gelatin solution from porcine skin, bloom 60 in 50 mM carbonate-bicarbonate buffer, pH9.6 at the concentration of 200 μl/well, for 2 hours at 37° C. The coating solution is carefully removed and the sample to be analyzed was added (100 μl/well) at the appropriate dilution. Dilutions of a human apoE standard are simultaneously assayed. Samples and antibodies are diluted in PBS, 1% BSA, 0.1% Tween 20, pH 7.4. Samples are incubated for 1 hour at 37° C. and the wells are washed 3 times with 200 μl of buffer solution. One hundred microliters per well of the primary antibody (goat anti-human apoE IgG) diluted 10000 fold is incubated for 1 hour at 37° C. under continuous shaking. After the third wash, 100 μl/well of the secondary antibody (anti-goat-IgG peroxidase conjugate) diluted 5000 fold is incubated for 1 hour at 37° C. with continuous shaking. Wells are washed 5 times and 100 μl/well of substrate (ortho-phenylenediamine dihydrochloride) is incubated for the appropriate time at room temperature in the dark with continuous shaking. The reaction is stopped by adding 50 μl/well of 3M sulfuric acid and incubating for 1 min. at room temperature. The absorbance at 492 nm versus 620 nm is read on a microplate photometer and the results are then converted in human apoE ng equivalent.

This assay was validated by testing ATRA (all-trans retinoic acid) as reference compound. Test results showed that ATRA decreased the production of apoE by THP-1 cells, which is consistent with decreased apoE plasma levels observed in vivo in the rat. Since ATRA is known to act by regulating the expression of specific genes controlled by nuclear receptors, THP-1 cells can be used as a relevant model to screen compounds affecting the expression of the apoE gene.

The present invention has been shown by both description and examples. The Examples are only examples and cannot be construed to limit the scope of the invention. One of ordinary skill in the art will envision equivalents to the inventive process described by the following claims that are within the scope and spirit of the claimed invention.

### REFERENCES

The following references, to the extent that they provide exemplary procedural or other details supplementary to those set forth herein, are specifically incorporated herein by reference.
Berge et al., “Pharmaceutical salts,” J. Pharm. Sci., 66:1-19, 1997.
Gaillard, “Apolipoprotein E intrathecal synthesis is decreased in multiple sclerosis patients,” Ann. Clin. Biochem., 33:148-150,1996.
Goodrum & Boudin, “The cell biology of myelin and regeneration in the peripheral nervous system,” J. Neuropathol.Exp.Neurol. 55:943-953, 1996.
Laskowitz et al., “Apolipoprotein E-deficient mice have increased susceptibility to focal cerebral ischemia,” J. Cerebral Blood Flow Metab., 17: 753-758 1997.
Leininger-Muller & Siest, “The rat, a useful model for pharmacological studies on apolipoprotein E,” Life Sciences, 58:455-467, 1996.
Linton et al., “Prevention of atherosclerosis in apolipoprotein E-deficient mice by bone marrow transplantation” Science, 267:1034-1037, 1995.
Oitzl et al., “Severe learning deficits in apolipoprotein E knockout mice in a water maze task,” Brain Res., 752:189-196, 1997.
Poirier, “Apolipoprotein E in animal models of CNS injury and in Alzheimer's disease,” Trends in Neurosciences 17:525-530, 1994.
Plump, “Atherosclerosis and the mouse—a decade of experience,” Annal. Med., 29:193-198, 1997.
Richard et al., “Identification of a new apolipoprotein E variant (E(2) Arg(142→Leu) in type III hyperlipidemia” Atherosclerosis, 112:19-28, 1995.
Roselaar & Daugherty, “Apolipoprotein E-deficient mice have impaired innate immune responses to listeria monocytogenes in vivo,” J. Lipid Res., 39:1740-1743, 1998.
Rubinsztein, “Apolipoprotein E—a review of its roles in lipoprotein metabolism, neuronal growth and repair and as a risk factor for Alzheimer's disease,” Psychological Med., 25:223-229, 1995.
Srivastava et al., “Estrogen up-regulates apolipoprotein E (ApoE) gene expression by increasing apoE MRNA in the translating pool via the estrogen receptor alpha-mediated pathway” J. Biol. Chem., 272:33360-33366, 1997.
Stone et al., “Astrocytes and microglia respond to estrogen with increased apoE mRNA in vivo and in vitro” Experimental Neurology, 143:313-318, 1997.
van Vlijmen et al., “Apolipoprotein E*3 Leiden transgenic mice as a test model for hypolipidaemic drugs,” Arzneimittel-Forschung, 48:396-402, 1998.
Walker et al., “Cerebral lipid deposition in aged Apolipoprotein E-deficient mice”, Am. J. Pathol., 151:1371-1377, 1997.

1. A method of modulating the production of apoE by an apoE producing cell, comprising contacting said apoE producing cell with an effective amount of a compound of formula (I): 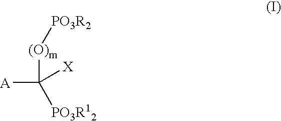
wherein X is H, OH, OCOCH₃ or NH₂;
R and R¹ are the same or different and are selected from H, CH₃ or C₂H₇, n- or i-C₃H₅,
n-, i- or s-C₄H₉;
m is zero or 1;
A is selected from: 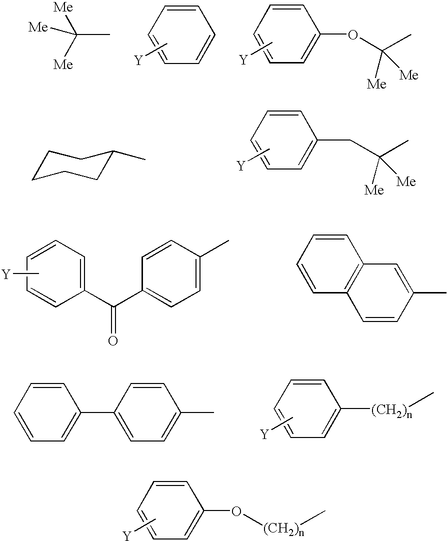
where n is an integer from 1 to 6 and Y is H, CH₃, OCH₃, CF₃, Br or Cl;
or a pharmaceutically acceptable salt thereof.

2. The method of claim 1, wherein X is OH and m is zero.

3. The method of claim 1, wherein X is H and m is 1.

4. The method of claim 1, wherein X is H and m is zero.

5. The method of claim 1, wherein said compound of formula (I) is selected from the group consisting of:
dimethyl α-(dimethoxyphosphinyl)-p-chlorobenzyl phosphate;
dimethyl α-(dimethoxyphosphinyl)-o-chlorobenzyl phosphate;
dimethyl α-(dimethoxyphosphinyl)-m-chlorobenzyl phosphate;
dimethyl α-(dimethoxyphosphinyl)-2,4-dichlorobenzyl phosphate;
diethyl α-(diethoxyphosphinyl)-p-chlorobenzyl phosphate;
diisopropyl α-(diisopropoxyphosphinyl)-p-chlorobenzyl phosphate;
dipropyl α-(dipropyloxyphosphinyl)-p-chlorobenzyl phosphate;
dibutyl α-(dibutyloxyphosphinyl)-p-chlorobenzyl phosphate;
dimethyl α-(dimethoxyphosphinyl)-benzyl phosphate;
dimethyl α-(dimethoxyphosphinyl)-p-methylbenzyl phosphate;
dimethyl α-(dimethoxyphosphinyl)-p-methoxybenzyl phosphate;
dimethyl α-(dimethoxyphosphinyl)-p-fluorobenzyl phosphate;
dimethyl α-(dimethoxyphosphinyl)-m-bromobenzyl phosphate;
dimethyl α-(dimethoxyphosphinyl)-p-(trifluoromethyl)benzyl phosphate,
dimethyl α-(dimethoxyphospbinyl)-4-(4-chlorobenzoyl)benzyl phosphate;
dimethyl [1-(dimethoxyphosphinyl)-2,2-dimethyl-2-phenyl]ethyl phosphate;
dimethyl [1-(dimethoxyphosphinyl)-2,2-dimethyl-2-(p-chlorophenyl)]ethyl phosphate;
dimethyl [1-(dimethoxyphosphinyl3-2,2-dimethyl-2-benzyl]ethyl phosphate;
dimethyl α-(dimethoxyphosphinyl)cyclohexylmethyl phosphate;
dimethyl α-(dimethoxypbosphinyl)-2-naphthylmethyl phosphate;
dimethyl α-(dimethoxyphosphinyl)-4-biphenylmethyl phosphate;
tetraethyl 2-(p-chlorophenylethylidene)1,1-diphosphonate;
tetrabutyl 2-(p-chlorophenylethylidene)1,1-diphosphonate;
tetramethyl 2-(p-chlorophenylethylidene) 1,1 -diphosphonate;
tetramethyl 1-chloro-2-(p-chlorophenylethylidene)1,1-diphosphonate;
tetraethyl 2-(phenylethylidene)1,1-diphosphonate;
tetraethyl 3-(phenylpropylidene)1,1-diphosphonate;
tetraethyl 4-(phenylbutylidene)1,1-diphosphonate;
tetraethyl 3-(phenoxypropylidene)1,1-diphosphonate;
tetraethyl 4-(phenoxybutylidene)1,1-diphosphonate;
tetramethyl 2-(phenylethylidene)1,1-diphosphonate;
tetramethyl 3-(phenylpropylidene)1,1-diphosphonate;
tetramethyl 4-(phenylbutylidene)1,1-diphosphonate;
tetramethyl 3-(phenoxypropylidene)1,1-diphosphonate; and
tetramethyl 4-(phenoxybutylidene)1,1-diphosphonate.

6. The method of claim 1, wherein said modulation of the production of apoE by said apoE producing cell comprises increasing the production of apoE by said apoE producing cell.

7. The method of claim 1, wherein said modulation of the production of apoE by said apoE producing cell comprises decreasing the production of apoE by said apoE producing cell.

8. A method of modulating apoE levels in a patient in need of such treatment, comprising administration of an effective amount of a compound of formula (I): 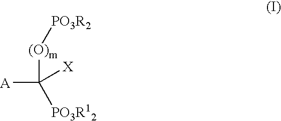
wherein X is H, OH, OCOCH₃ or NH₂;
R and R¹ are the same or different and are selected from H, CH₃ or C₂H₇, n- or i-C₃H₅,
n-, i- or s-C₄H₉;
m is zero or 1;
A is selected from: 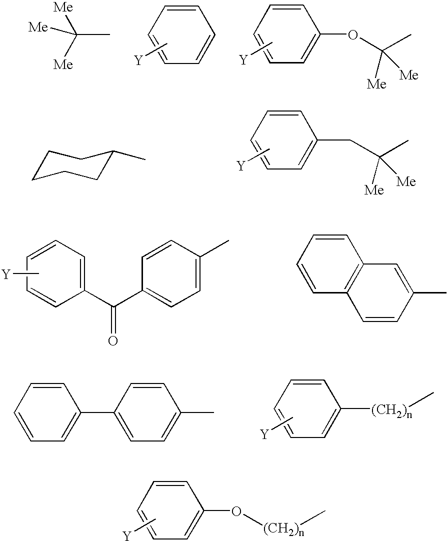
where n is an integer from 1 to 6 and Y is H, CH₃, OCH₃, CF₃, Br or Cl; or a pharmaceutically acceptable salt thereof.

9. The method of claim 8, wherein X is OH and m is zero.

10. The method of claim 8, wherein X is H and m is 1.

11. The method of claim 8, wherein X is H and m is zero.

12. The method of claim 8, wherein said compound of formula (I) is selected from the group consisting of:
dimethyl α-(dimethoxyphosphinyl)-p-chlorobenzyl phosphate;
dimethyl α-(dimethoxyphosphinyl)-o-chlorobenzyl phosphate;
dimethyl α-(dimethoxyphosphinyl)-m-chlorobenzyl phosphate;
dimethyl α-(dimethoxyphosphinyl)-2,4-dichlorobenzyl phosphate;
diethyl α-(diethoxyphosphinyl)-p-chlorobenzyl phosphate;
diisopropyl α-(diisopropoxyphosphinyl)-p-chlorobenzyl phosphate;
dipropyl α-(dipropyloxyphosphinyl)-p-chlorobenzyl phosphate;
dibutyl α-(dibutyloxyphosphinyl)-p-chlorobenzyl phosphate;
dimethyl α-(dimethoxyphosphinyl)-benzyl phosphate;
dimethyl α-(dimethoxyphosphinyl)-p-methylbenzyl phosphate;
dimethyl α-(dimethoxyphosphinyl)-p-methoxybenzyl phosphate;
dimethyl α-(dimethoxyphosphinyl)-p-fluorobenzyl phosphate;
dimethyl α-(dimethoxyphosphinyl)-m-bromobenzyl phosphate;
dimethyl α-(dimethoxyphosphinyl)-p-(trifluoromethyl)benzyl phosphate,
dimethyl α-(dimethoxyphosphinyl)-4-(4-chlorobenzoyl)benzyl phosphate;
dimethyl [1-(dimethoxyphosphinyl)-2,2-dimethyl-2-phenyl]ethyl phosphate;
dimethyl [1-(dimethoxyphosphinyl)-2,2-dimethyl-2-(p-chlorophenyl)]ethyl phosphate;
dimethyl [1-(dimethoxyphosphinyl)-2,2-dimethyl-2-benzyl]ethyl phosphate;
dimethyl α-(dimethoxyphosphinyl)cyclohexylmethyl phosphate;
dimethyl α-(dimethoxyphosphinyl)-2-naphthylmethyl phosphate;
dimethyl α-(dimethoxyphosphinyl)-4-biphenylmethyl phosphate;
tetraethyl 2-(p-chlorophenylethylidene)1,1-diphosphonate;
tetrabutyl 2-(p-chlorophenylethylidene)1,1-diphosphonate;
tetramethyl 2-(p-chlorophenylethylidene)1,1-diphosphonate;
tetramethyl 1-chloro-2-(p-chlorophenylethylidene)1,1-diphosphonate;
tetraethyl 2-(phenylethylidene)1,1-diphosphonate;
tetraethyl 3-(phenylpropylidene)1,1-diphosphonate;
tetraethyl 4-(phenylbutylidene)1,1-diphosphonate;
tetraethyl 3-(phenoxypropylidene)1,1-diphosphonate;
tetraethyl 4-(phenoxybutylidene)1,1-diphosphonate;
tetramethyl 2-(phenylethylidene)1,1-diphosphonate;
tetramethyl 3-(phenylpropylidene)1,1-diphosphonate;
tetramethyl 4-(phenylbutylidene)1,1-diphosphonate;
tetramethyl 3-(phenoxypropylidene)1,1-diphosphonate; and
tetramethyl 4-(phenoxybutylidene)1,1-diphosphonate.

13. The method of claim 8, wherein said modulation of said apoE levels in said patient comprises increasing said apoE levels.

14. The method of claim 13, wherein said patient is suffering from atherosclerosis, Alzheimer's disease, macular degeneration, retinitis pigmentosa, stroke, degenerative neuropathy, xanthoma or xanthelasma.

15. The method of claim 14, wherein said degenerative neuropathy is associated with diabetic neuropathy or multiple sclerosis.

16. A method of elevating high density cholesterol, comprising administration of a compound of formula (I) according to claim 13.

17. A method for preventing and/or treating atherosclerosis, comprising administration of a compound of formula (I) according to claim 13.

18. A method for preventing and/or treating macular degeneration and retinitis pigmentosa comprising, administration of a compound of formula (I) according to claim 13.

19. A method for the preventing and/or treating stroke, comprising administration of a compound of formula (I) according to claim 13.

20. A method for the prevention of degenerative neuropathy, comprising administration to a compound of formula (I) according to claim 13.

21. The method of claim 20, wherein said degenerative neuropathy is associated with diabetic neuropathy or multiple sclerosis.

22. The method of claim 8, wherein said modulation of said apoE levels in said patient comprises decreasing said apoE levels.

23. The method of claim 22, wherein said patient expresses apoE4, apoE Leiden or a non-functional mutant form of apoE.

24. The method of claim 22, wherein said patient is suffering from atherosclerosis or Alzheimer's disease.

25. A method for the prevention and/or treatment of Alzheimer's disease or dementia comprising administration to a patient an effective amount of a compound of formula (1) as claimed in claim 1.

26. The method of claim 25, wherein said patient is heterozygous or homozygous for apoE2 and/or apoE3 and wherein said compound of formula (I) increases apoE levels in said patient.

27. The method of claim 25, wherein said patient is heterozygous or homozygous for apoE4 and said compound of formula (I) decreases apoE levels in said patient.
